(19) United States
(12) Patent Application Publication
(10) Pub. No.: US 2015/0273156 A1
(43) Pub. Date: Oct. 1, 2015

(54) DEVICES FOR SYSTEMIC DRUG DELIVERY AND RELATED METHODS OF USE

(71) Applicant: Boston Scientific Scimed, Inc., Maple Grove, MN (US)

(72) Inventors: Javier PALOMAR-MORENO, Galway (IE); Martyn G. FOLAN, Galway (IE); Fergal HORGAN, Co. Mayo (IE); Michael G. HAYES, Galway (IE); Patricia KELLY, Galway (IE)

(73) Assignee: Boston Scientific Scimed, Inc.

(21) Appl. No.: 14/668,066

(22) Filed: Mar. 25, 2015

Related U.S. Application Data
(60) Provisional application No. 61/970,446, filed on Mar. 26, 2014.

(51) Int. Cl. A61M 5/315; A61L 31/16; A61L 31/04; A61M 5/44

(52) U.S. Cl. CPC A61M 5/31528; A61M 5/445; A61L 31/16; A61L 31/04

(57) ABSTRACT

A method of treating a disease of the lung may include inserting an implant into the circulatory system. The implant may include an agent configured to be released into the circulatory system over time to deliver the agent to the lungs. The agent may be configured to treat a disease of the lung.

### CROSS-REFERENCE TO RELATED APPLICATION

This patent application claims the benefit of priority to U.S. Provisional Patent Application No. 61/970,446, filed on Mar. 26, 2014, the entirety of which is incorporated herein by reference.

### TECHNICAL FIELD

Various embodiments of the present disclosure relate generally to devices for systemic drug delivery and related methods of use. More specifically, the present disclosure relates to systemic delivery of drugs for treating the lung.

### BACKGROUND

Chronic obstructive pulmonary disease (COPD) includes conditions such as, e.g., chronic bronchitis and emphysema. These conditions are often co-existing within patients having COPD. COPD currently affects over 15 million people in the United States alone and is currently the third leading cause of death in the country. The primary cause of COPD is inhalation of cigarette smoke, responsible for over 90% of COPD cases. The economic and social burden of the disease is substantial and is increasing. Other diseases of the lung include asthma, allergies, and cancer, among others.

Chronic bronchitis is characterized by chronic cough with sputum production. Due to airway inflammation, mucus hypersecretion, airway hyperresponsiveness, and eventual fibrosis of the airway walls, significant airflow and gas exchange limitations result. Chronic bronchitis can lead to a blockage of the airways and debilitating exacerbative episodes that can pose serious health risks to COPD patients.

Emphysema is characterized by the destruction of the lung parenchyma. This destruction of the lung parenchyma leads to a loss of elastic recoil and tethering which maintains airway patency. Because bronchioles are not supported by cartilage like the larger airways, they have little intrinsic support and therefore are susceptible to collapse when destruction of tethering occurs, particularly during exhalation.

Strategies for managing COPD include smoking cessation, vaccination, rehabilitation, and drug treatments (e.g., inhalers or oral medication). Drug treatments of COPD conditions, such as, e.g., mucus production, often suffer from poor patient compliance. That is, certain patients may not accurately administer prescribed doses, reducing the efficacy of treatment. For drug treatments utilizing inhalation, there is also an accompanying drug loss due to upper airway entrapment, which may lead to an over-prescription of active drugs. For drug treatments utilizing oral administration, there is an accompanying systemic loss which also leads to an over-prescription of active drugs. The over-prescription of drugs may result in suboptimal treatment and/or a build-up of toxins within the lungs and/or other organ systems.

Thus, a need exists for drug delivery mechanisms that efficiently and effectively treat diseases of the lungs.

### SUMMARY OF THE DISCLOSURE

The present disclosure includes devices for systemic drug delivery and related methods of use.

In one aspect, the present disclosure is directed to a method of treating a disease of the lung. The method may include inserting an implant into the circulatory system. The implant may include an agent configured to be released into the circulatory system over time to deliver the agent to the lungs. The agent may be configured to treat a disease of the lung.

Various embodiments of the present disclosure may also include one or more of the following aspects: wherein the agent may be released into the bloodstream, and the agent may enter the lung through an exchange between one or more capillaries of the circulatory system and one or more alveoli of the lung; wherein the agent may be configured to treat one or more of COPD, bronchitis, emphysema, asthma, an allergy of the lung, and lung cancer; wherein the implant may be configured to gradually degrade over a period of months to release the agent; wherein the implant may be inserted into a vein that leads to the right atrium of the heart; wherein the implant may be polymeric; and wherein inserting the implant into the circulatory system may further include the implant coupled to an expandable member, the expandable member being movable between a retracted configuration and an expanded configuration, inserting the expandable member and the implant into the circulatory system while the expandable member is in the retracted configuration, and expanding the expandable member to the expanded configuration to adhere the implant to an inner surface in the circulatory system.

In another aspect, the present disclosure is directed to a medical device. The medical device may include a first elongate member having a lumen extending between a proximal end and a distal end of the first elongate member. The medical device may also include a second elongate member disposed through the first elongate member. The second elongate member may include a handle portion disposed at a proximal end, and a lumen disposed through the second elongate member and the handle portion. The medical device may also include a plunger disposed in the second elongate member. The plunger may include a proximal portion, and a third elongate member extending distally from the proximal portion. The medical device may also include an implant disposed within the second elongate member distal to the plunger, wherein the implant includes an agent configured to be released into the circulatory system over time to deliver the agent to the lungs. The agent may be configured to treat a disease of the lung.

Various embodiments of the present disclosure may also include one or more of the following aspects: further including a flange disposed at the proximal end of the first elongate member, wherein, in a first configuration, the second elongate member may be disposed at a proximal position such that a distal end of the second elongate member is disposed within the first elongate member, and the handle portion of the second elongate member is disposed proximal to the flange of the first elongate member by a first distance; wherein, in a second configuration, the second elongate member may be disposed at a distal position such that the distal end of the second elongate member is disposed distal to the distal end of the first elongate member, an entirety of the implant is disposed distal to the distal end of the first elongate member, the second elongate member, the plunger, and the implant are disposed distally by approximately the first distance relative to their respective positions in the first configuration, and the handle portion of the second elongate member and the flange of the first elongate member are adjacent to one another; wherein, in a third configuration the plunger and the implant may be disposed in the substantially same positions as the second configuration, and the second elongate member is disposed proximal to the distal position of the second elongate member to remove the implant from the distal end of the second elongate member; and wherein the plunger further includes a stopper disposed at a distal end of the third elongate member.

In yet another aspect, the present disclosure may be directed to a medical device. The medical device may include a first elongate member having a lumen extending between a proximal end and a distal end, and a fluid delivery device containing an agent. The agent may be configured to be dispensed into the circulatory system over time to deliver the agent to the lungs, the agent may be configured to treat a disease of the lung. The medical device may include an actuator coupled to the first elongate member and the fluid delivery device, and a distal tip coupled to the fluid delivery device. The proximal movement of the actuator may move the distal tip longitudinally and dispense agent from the distal tip.

Various embodiments of the present disclosure may also include one or more of the following aspects: further including a heater that may be coupled to the fluid delivery device configured to maintain the agent in liquid form; wherein the fluid delivery device may further contain a matrix, and wherein the matrix and the agent may be configured to be in solid form at a human body temperature; wherein the proximal movement of the actuator may move the distal tip along and about a longitudinal axis in a spiral path; wherein the actuator may be an elongate member having a thread, and the first elongate member may further include a bore configured to receive the actuator; further including a drive member coupled to a proximal end of the actuator, wherein the proximal movement of the actuator causes the drive member to dispense the agent from the fluid delivery device and through the distal tip; further including a conduit that may extend from the fluid delivery device, through the first elongate member, and in fluid communication with the distal tip; wherein in a first configuration, the distal tip may be constrained within the first elongate member, and the first elongate member may be configured to retract relative to the distal tip to remove the distal tip from the first elongate member in a second configuration; and wherein the actuator may include an elongate member having an elongate thread, the medical device may further include a fastener configured to receive the actuator, and the conduit may further extend through the actuator.

### BRIEF DESCRIPTION OF THE FIGURES

The accompanying drawings, which are incorporated in and constitute a part of this specification, illustrate various exemplary embodiments and together with the description, serve to explain the principles of the disclosed embodiments.

FIG. 1 is an illustration of portions of the circulatory system, heart, and respiratory system having a drug delivery implant in accordance with an embodiment of the present disclosure.

FIG. 2 is an illustration of gas and drug exchange between the circulatory and respiratory systems in accordance with an embodiment of the present disclosure.

FIGS. 3-6 illustrate a drug delivery device and method of delivering a drug delivery implant in accordance with an embodiment of the present disclosure.

FIGS. 7-9 illustrate a drug delivery device and method of delivering a drug delivery implant in accordance with another embodiment of the present disclosure.

FIGS. 10-11 illustrate a drug delivery device and a method of delivering a drug delivery implant in accordance with another embodiment of the present disclosure.

FIG. 12 is a partial side view of a drug delivery device in accordance with an embodiment of the present disclosure.

FIG. 13 is a cross-sectional view of the drug delivery device of FIG. 12 taken along line 13-13.

### DETAILED DESCRIPTION

Reference will now be made in detail to embodiments of the present disclosure, examples of which are illustrated in the accompanying drawings. Wherever possible, the same reference numbers will be used throughout the drawings to refer to the same or like parts.

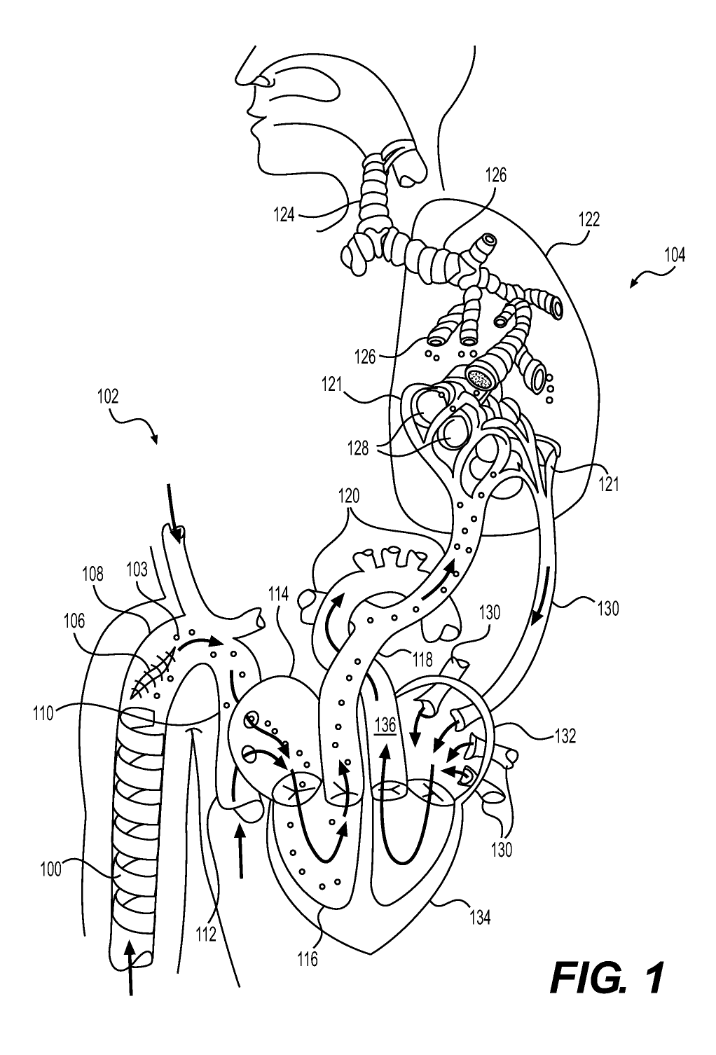

FIG. 1 illustrates an exemplary implant 100 that may be inserted within the circulatory system 102 of a patient, to deliver an agent 103, such as e.g., a drug, to the respiratory system 104. Implant 100 may be delivered to the circulatory system 102 through an incision 106 of a vein 108. Incision 106 may be subsequently sutured or otherwise closed after insertion of implant 100. In one embodiment, vein 108 may a subclavian vein that is a continuation of the axillary vein running from an outer border of a rib to the medial border of the anterior scalene muscle. However, it should be noted that vein 108 may be any other vein suitable for receiving an implant 100.

Blood may flow through vein 108, carrying agent 103 toward superior vena cava 110. While implant 100 is depicted as being placed within a vein 108 that flows to superior vena cava 110, it should be noted that implant 100 may additionally or alternatively be placed within a vein 108 that flows to inferior vena cava 112. Blood may carry agent 103 from the superior vena cava 110 and/or inferior vena cava 112 toward the right atrium 114 of the heart, where agent 103 may be pumped to right ventricle 116.

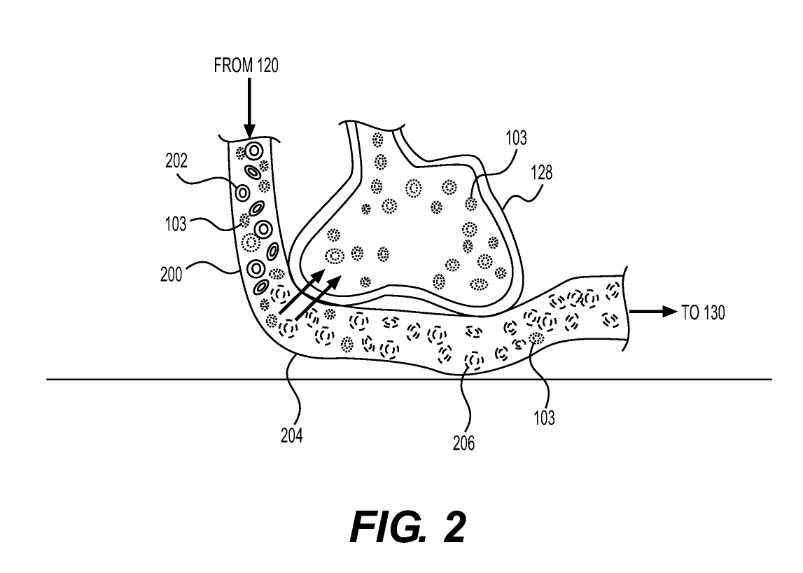

Blood may flow from right ventricle 116 and carry agent 103 toward pulmonary trunk 118 that divides into pulmonary arteries 120. Pulmonary arteries 120 may divide into a number of segments 121 that form a plurality of capillaries 204 (shown only in FIG. 2). The capillaries 204 may serve as an interface between circulatory system 102 and respiratory system 104 that includes a lung 122.

A trachea 124 may allow for the passage of air toward airways 126 of lung 122. Airways 126 may include a network of airways that branch throughout lung 122. Airways 126 may include the main bronchi and subsequent generations of bronchioles that eventually terminate distally into a plurality of alveoli 128. Alveoli 128 may be located within the lung parenchyma, and may be the terminal distal ends of the respiratory system 104. Alveoli 128, along with the capillaries of the circulatory system 102 (described in further detail with respect to FIG. 2) form a gas-exchange surface between circulatory system 102 and respiratory system 104.

After gas exchange with the alveoli, oxygenated blood that may have little or no agent 103 may flow from lung 122 through one of a plurality of pulmonary veins 130 toward left atrium 132 of the heart. From left atrium 132, blood may be pumped through left ventricle 134, and may exit the heart through aorta 136. From aorta 136, blood may be pumped through the body, and return to the heart via vein 108 having implant 100, so that the blood may carry additional agent 103 toward lungs 122. Thus, because circulatory system 102 continuously circulates blood through the body, a continuous supply of agent 103 may be delivered to lungs 122 until the supply of agent 103 is depleted.

Additionally or alternatively, implant 100 may be placed directly within pulmonary trunk 118, one or more pulmonary arteries 120, segments 121, and/or capillaries 204 by a suitable procedure to deliver agent 103 to lung 122.

FIG. 2 is an illustration of the gas-exchange interface between circulatory system 102 and respiratory system 104. Deoxygenated blood 202 and agent 103 may flow from pulmonary arteries 120 to one of a plurality of capillaries 204 that are interfaced with alveoli 128. Carbon dioxide (not shown) and active agent 103 may flow from deoxygenated blood 202 in capillaries 204 into alveoli 128, while oxygen (not shown) may flow from alveoli 128 into capillaries 204 so that blood may leave the gas-exchange interface as oxygenated blood 206. It should be noted that while it is anticipated that a significant amount of agent 103 circulating through the blood will be delivered into alveoli 128, some agent 103 may continue to flow through the body with oxygenated blood 204 toward pulmonary vein 130. From alveoli 128, agent 103 may travel proximally through airways 126 and/or parenchymal tissue of the lung to treat one or more diseases of the lung.

With continued reference to FIG. 1, implant 100 may a resorbable implant that is configured to be broken down and assimilated into the body of a patient (e.g., biodegradable) over a period of time. In some embodiments, implant 100 may deliver drugs to the lungs for a period of four to six months, although other time periods for drug delivery, both lesser and greater, are also contemplated. Implant 100 may include a matrix mixed with agent 103. The matrix may include polymers or other materials such as, e.g., polylactic acid, polyglycolic acid (PGA), collagen or other connective proteins or natural materials, polycaprolactone, hylauric acid, and/or adhesive proteins. In addition, the matrix may include co-polymers, composites, and combinations of these and other suitable biodegradable materials. In some embodiments, polyester and/or polycarbonate co-polymers may be utilized in the matrix. In yet another embodiment, the matrix may include the co-polymer poly(lactic-co-glycolic acid) (PLGA) having a molecular weight of about 65 kDa as a copolymer of about 85% lactide and 15% glycolide. In some embodiments, agent 103 may be incorporated into microparticles, nanoparticles, or other suitable particles that are configured to embed within lung tissue and subsequently elute agent 103.

Agent 103 may be mixed within the matrix in solution. Agent 103 also may be mixed in similar methods utilized for drug eluting stent (DES) coatings. In some embodiments, agent 103 may be additionally or alternatively coated onto an outer surface of implant 100. Agent 103 may be selected from the family of oral or inhaled medications currently available for treatment of COPD, asthma, lung cancer and/or other conditions of the lung. Additionally, agent 103 may be used to treat any other condition of the body. In one exemplary embodiment, agent 103 may be utilized to deliver a vaccine (e.g., a flu vaccine) over a period of multiple months (e.g., the winter season) to a patient. Agent 103 may be released into the bloodstream by the hydrolysis of the matrix of implant 100. In some embodiments, agent 103 may include bronchodilators, inhaled steroids, oral steroids, phosphodiesterase-4 inhibitors (e.g., roflumilast), theophylline, antibiotics, or any suitable combination. Bronchodilators, which may otherwise be delivered by inhalation, may include long-acting bronchodilators (tiotropium, salmeterol, formoterol, arformoterol, indacaterol, aclidinium, and the like) or short-acting bronchodilators (albuterol, levalbuterol, ipratropium, and the like). Inhaled steroids may include inhaled corticosteroids that may reduce airway inflammation and help prevent exacerbations (fluticasone, budesonide, and the like). Exemplary combinations of drugs include salmeterol and fluticasone, and formoterol and budesonide. Other agents, such as, e.g., carbocisteine, mecysteine, N-acetylcysteine, may be additionally or alternatively utilized. Agent 103 may also include agents directed toward asthma, allergies, cancers, or other ailments of the lung.

In some embodiments, implant 100 may be helical, although other suitable shapes, such as, e.g., cylindrical, woven, are also contemplated. An outer surface of implant 100 may also include a bioadhesive material for facilitating implantation within, e.g., vein 108. The bioadhesive material may include natural polymeric materials, synthetic materials, and/or synthetic materials formed from biological monomers such as sugars. The bioadhesive material may be obtained from the secretions of microbes or by marine mollusks and crustaceans. The bioadhesive material may be designed to adhere to biological tissue. In at least one embodiment, the adhesive activity of the adhesive layer may be controlled through compound design such that an exposure time is required for tracking the device to the vascular location before the adhesive is ready to bond to the lumen wall.

In some embodiments, the bioadhesive material(s) may include, but are not limited to, amino adhesives, adhesive surface proteins (MSCRAMMS), adhesively modified biodegradable polymers such as Fatty Ester Modified PLA/PLGA, polymer materials, minigel particles, or other suitable bioadhesives. The bioadhesive material may be dissolved in a solvent or co-solvent blend prior to application to the outer surface of implant 100. The solvent may include alcohols (e.g., methanol, ethanol, and isopropanol), water, or another suitable solvent.

Amino acid bioadhesives may be utilized to facilitate adhesion of implant 100 to a target location (e.g., a lesion site in vein 108). Zwitterionic amino acids may be employed as a layer or as a component within implant 100. The zwitterionic amino acid may be oriented so that the hydrophobic side of the zwitterionic amino acid selectively facilitates adhesion to the lipophilic vascular wall. In one embodiment, the amino acid 3,4-L-dihydroxyphenylalanine (DOPA), which is a tyrosine derivative found in high concentrations in the “glue” proteins of mussels, may be utilized.

MSCRAMMs (microbial surface components recognizing adhesive matrix molecules) may be employed as a bioadhesive. MSCRAMMS may include materials naturally-produced by pathogens to initiate adhesion to the host extracellular matrix to initiate infection. These adhesive surface proteins may be isolated or synthesized, and utilized to facilitate adhesion of implant 100 to a target location within vein 108.

Adhesively modified biodegradable polymers may include DOPA (L-3,4-dihydroxyphenylalanine) modified PLA (polylactic acid), PLGA poly(lactide-co-glycolide), among others. In such embodiments, examples of suitable adhesive moieties include, but are not limited to, monopalmitate, monostearin, glycerol, dilaurin, iso-stearyl alcohol, or the like.

Other polymer materials may alternatively be utilized as bioadhesives, including, but not limited to, proteins (e.g., gelatin) and carbohydrates (e.g., starch). For example, polysaccharides such as sorbitol, sucrose, xylitol, anionic hydrated polysaccharides (gellan, curdlan, XM-6, and xanthan) may also be employed as a bioadhesive. Other suitable materials include derivatives of natural compositions such as algenic acid, hydrated gels and the like, and also biocompatable polymers and oligomers such as dextrans, dextranes, dextrins, hydrogels including, but not limited to, polyethylene glycol (PEG), polyethylene glycol/dextran aldehyde, polyethylene oxide, polypropyline oxide, polyvinylpyrrolidine, polyvinyl acetate, polyhydroxyethyl methacrylate, and polyvinyl alcohol, as well as derivatives thereof may also be employed herein.

Minigel particles may additionally or alternatively be utilized as a bioadhesive. One exemplary bioadhesive is poly(NIPAM) (poly(N-isopropylacrylamide) minigel particles. Poly(NIPAM) may be in a liquid state at room temperature, and an adhesive at body temperature. Additionally, for improved retention of the polymer on the surface of implant 100, minigel particles may be crosslinked or mixed with a higher molecular weight polymer to allow enough time for retention of the minigel to the medical device during delivery, or uncrosslinked minigel particles can be employed in a crosslinked polymer network.

In an alternative embodiment, implant 100 may utilize anchors, such as, e.g., hooks, barbs, and the like to adhere to the surface of vein 108. In some embodiments, the anchors may be biodegradable and be formed of the same material as the remaining portion of implant 100, or may be formed of another suitable material.

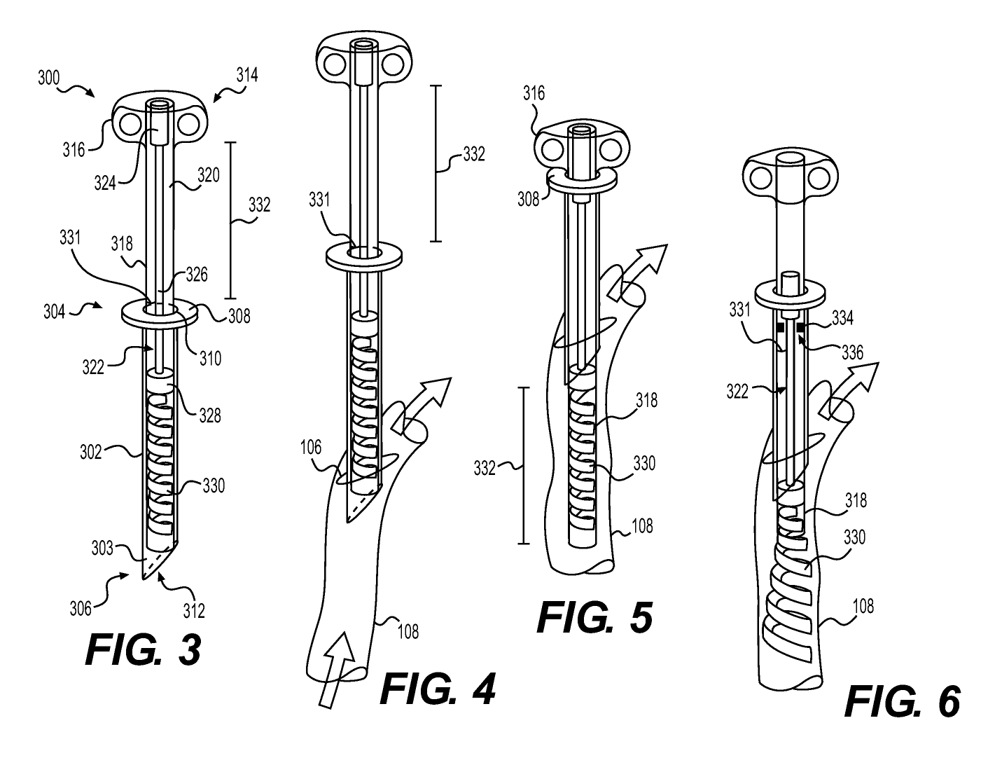

A medical device 300 configured to deliver an implant 330 is depicted in FIGS. 3-6. Implant 330 may be substantially similar to implant 100 described with reference to FIG. 1. Medical device 300 may include an elongate member 302 having a lumen 303 that extends from a proximal end 304 toward a distal end 306 of elongate member 302. A flange 308 may be disposed at proximal end 304 of elongate member 302. An opening 310 in communication with lumen 303 may be disposed within flange 308. Elongate member 302 may also include an opening 312 disposed at distal end 306. Thus, openings 310 and 306 may be in communication with one another via lumen 303. Distal end 306 of elongate member 302 may be beveled so that elongate member 302 can pierce through tissue. In some embodiments, elongate member 302 may be a needle or other suitable object configured to deliver or withdraw fluids or other substances. Elongate member 302 may be formed of stainless steel or other metals, polymers, or other suitable materials.

Medical device 300 may further include an actuating assembly 314. A handle 316 may be disposed at the proximal end of actuating assembly 314. Actuating assembly 314 may also include an elongate member 318 extending distally from handle 316. A lumen 320 may be disposed through both handle 316 and elongate member 318. Elongate member 318 may have a length that is greater than the length of elongate member 302. Further, elongate member 318 may have a diameter that is lesser than or equal to the diameter of lumen 303 such that elongate member 318 can be slidably disposed within lumen 303 of elongate member 302.

A plunger 322 may be disposed within lumen 320, and may include a proximal portion 324, an elongate member 326, and a stopper 328. Proximal portion 324 may be compressible and/or atraumatic such that when actuating assembly 314 is moved distally, an operator may apply a force to proximal portion 324, with, e.g., a thumb or finger, without injury. Elongate member 326 may extend distally from proximal portion 324, and stopper 328 may be coupled to the distal end of elongate member 326. Stopper 328 may be configured to both slide within lumen 320, and form a seal with an interior surface of elongate member 318. Implant 330 may be disposed distal to plunger 322 within lumen 320 of actuating assembly 314.

As seen in FIG. 4, medical device 300 may be configured to deliver implant 330 to a vein 108 described with reference to FIG. 1. An operator may pierce through the tissue (not shown) of a patient with the beveled distal end 306 of elongate member 302 to form incision 106 through a surface of vein 108. Alternatively, incision 106 may be formed by another mechanism, such as, e.g., mechanical cutting, ablation, or the like, prior to insertion of elongate member 302 into vein 108. In FIGS. 3 and 4, medical device 300 may be in a first configuration where distal end 306 of elongate member 302 and the distal end of elongate member 318 are generally aligned along a longitudinal axis of medical device 300. In the first configuration, elongate member 318 may extend proximally from proximal end 304 of elongate member 302 by a distance 332.

Once distal end 306 is disposed through incision 106 and within vein 108 as shown in FIG. 4, an operator may then move actuating assembly 314 distally through lumen 303 until handle 316 comes into contact with flange 308 (shown in FIG. 5). The distal movement of actuating assembly 314 may move medical device 300 from the first configuration to a second configuration shown in FIG. 5. In some embodiments, the entireties of actuating assembly 314 (e.g., handle 316, elongate member 318, lumen 320, and plunger 322) and implant 330 may move distally through lumen 303. A distal end of elongate member 318 may extend through opening 312 of elongate member 302 into vein 108. In the second configuration of medical device 300, an entirety or a substantial entirety of implant 330 may be disposed distally to distal end 306 of elongate member 302, yet enclosed by elongate member 318. In the second configuration, elongate member 318 may extend distally from distal end 306 of elongate member 302 by the distance 332. Further, in the second configuration, plunger 322 may have been displaced distally by the distance 332 such that proximal portion 324 is disposed longitudinally adjacent to flange 308, and stopper 328 is disposed longitudinally adjacent to distal end 306 of elongate member 302.

After handle 316 comes into contact with flange 308 and an entirety of implant 330 is disposed distal to distal end 306 of elongate member 302, an operator may retract handle 316 of actuating assembly 314 proximally (as seen in FIG. 6). The proximal movement of handle 316 may also cause elongate member 318 to move proximally. In some embodiments, plunger 322 may not be retracted proximally while handle 316 and elongate member 318 are retracted proximally. Thus, FIG. 6 may depict a third configuration of medical device 300 where plunger 322 and implant 330 are disposed in approximately the same longitudinal positions as in the second configuration, but handle 316 and elongate member 318 are disposed proximal to their longitudinal positions in the second configuration. As stopper 328 maintains the longitudinal position of implant 330, the proximal retraction of handle 316 and elongate member 318 may remove implant 330 from the distal end of elongate member 318.

In some embodiments, implant 330 may be substantially similar to implant 100 described with reference to FIG. 1. Implant 330 may also be a self-expanding stent that initially may be in a collapsed configuration within lumen 320 of elongate member 318. Implant 330 may also be formed as a coiled flat wire (of, e.g., stainless steel, laser-cut Nitinol, or a biodegradable material). Once displaced from the distal end of elongate member 318, implant 330 may expand radially outward and/or longitudinally outward. Handle 316 and elongate member 318 may be pulled proximally until the entirety of implant 330 has been displaced from elongate member 318 within vein 108.

In some embodiments, an operator may be able to ascertain that the entirety of implant 330 has been displaced into vein 108 based upon the tactile feedback (or lack thereof) experienced when each coil of implant 330 (e.g., when implant 330 is helical) is displaced from the distal end of elongate member 318. In some embodiments, a marking 331 may be disposed on an outer surface of elongate member 318 to provide a visual indication that handle 316 and elongate member 318 have been pulled proximally to a location ensuring that the entirety of implant 330 has been displaced from elongate member 318. That is, once medical device 300 is in the second configuration, an operator may pull handle 316 proximally until at least marking 331 can be visualized proximal to flange 308. This may ensure that implant 330 has been fully deployed within vein 108. The distance between marking 331 and handle 316 may be approximately equal to distance 332. In some embodiments, medical device 300 may include radiopaque markers and/or coatings that can be visualized by ultrasound or other suitable imaging mechanisms. The radiopaque markers and/or coatings may be visualized on an imaging device or display to verify during or after an implantation procedure that implant 330 has been properly deployed.

In some embodiments, medical device 300 may include mechanisms to ensure that plunger 322 is not left within vein 108. For example, stop 334 may be disposed within lumen 320 of elongate member 318. Stop 334 may be configured to allow elongate member 326 to pass through an opening 336 within the stop 334. However, the opening 336 may have a smaller diameter than proximal portion 324 of plunger 322. Thus, when proximal portion 324 comes into contact with stop 334, plunger 322 may be prevented from further distal movement. Alternatively, medical device 300 may include another suitable mechanism for preventing plunger 322 from being accidentally left within vein 108. In some embodiments, an operator may retrieve plunger 322 by inserting a suitable tool through opening 310, such as, e.g., a grasper.

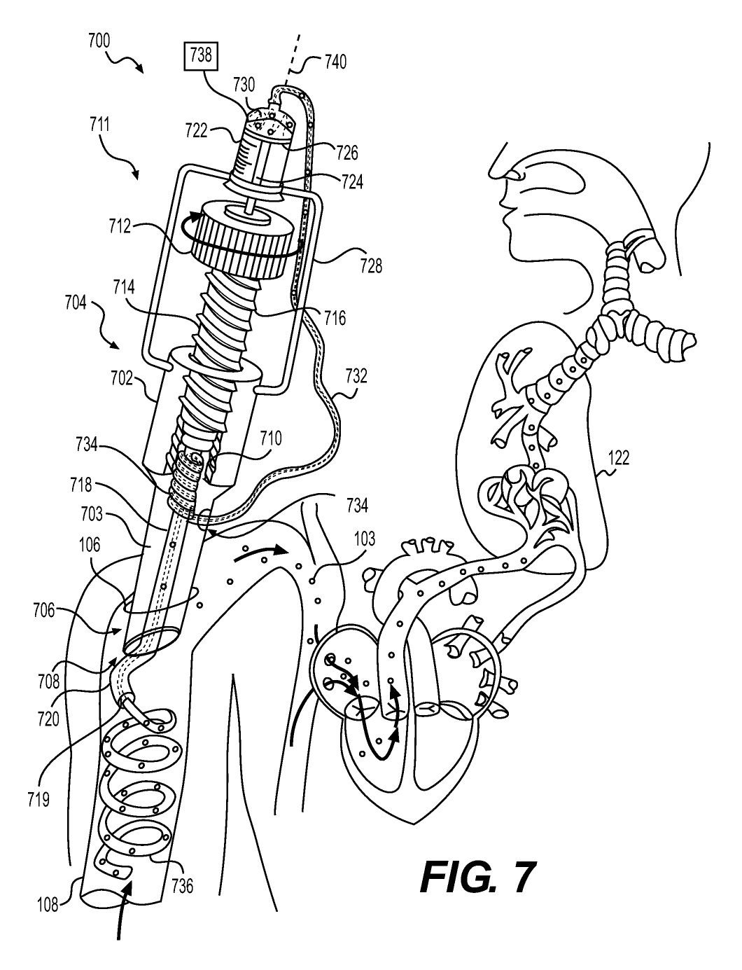

A medical device 700 configured to deliver an implant 736 is depicted in FIG. 7. Medical device 700 may include an elongate member 702 having a lumen 703 that extends from a proximal end 704 toward a distal end 706 of elongate member 702. Elongate member 702 may include an opening 708 disposed at distal end 706. Distal end 706 of elongate member 702 may be beveled so that elongate member 702 can pierce through tissue. In some embodiments, elongate member 702 may be a needle or other suitable object configured to deliver or withdraw fluids or other substances. Elongate member 702 may be formed from the similar materials as elongate member 302 described with reference to FIGS. 3-6.

Proximal end 704 of elongate member 702 may include a threaded bore 710. An actuating assembly 711 may include a handle 712 and an elongate member 714 extending distally from handle 712. Elongate member 714 may include a thread 716 configured to be inserted into threaded bore 710 of elongate member 702. While bore 710 is depicted as having a helical or spiral thread in FIG. 7, bore 710 may alternatively be formed without threads, or may include other suitable features for mating with various elongate members, such as, e.g., indents, tracks, protrusions, or the like.

A delivery member 718 may extend distally from elongate member 714 through lumen 703 of elongate member 702. Delivery member 718 may be a hollow lumen configured to deliver fluids or other suitable materials through a distal opening 719. Distal opening 719 may be disposed on a distal tip 720 of delivery member 718. Distal tip 720 may be helical, spiraled, and/or corkscrewed such that distal tip 720 wraps around longitudinal axis 740 of medical device 700. Alternatively, distal tip 720 may be formed in any suitable configuration. Thus, as delivery member 718 is moved along longitudinal axis 740, distal tip 720 may rotate about longitudinal axis 740.

A fluid delivery device 722, such as, e.g., a syringe, may extend proximally from handle 712 of actuating assembly 711 via a drive member 724. Fluid delivery device 722 may also be coupled to elongate member 702 via a support 728, such as, e.g., a bracket. Drive member 724 may be distally coupled to a stopper 726 that is configured to dispense a matrix 730 from fluid delivery device 722. In some embodiments, the proximal movement of handle 712 may cause drive member 724 and stopper 726 to move proximally through fluid delivery device 722 to dispense matrix 730 through a conduit 732. Conduit 732 may extend from a proximal end of fluid delivery device 722, through an opening 734 disposed in a side surface of elongate member 702, to a proximal end of delivery member 718.

Medical device 700 may be configured to deliver implant 736 to vein 108. Implant 736 may be a solidified form of matrix 730. That is, while disposed within fluid delivery device 722, matrix 730 may be heated and maintained as a liquid by a heater 738. Matrix 730 may solidify at a body temperature (e.g., approximately 37 degrees Celsius) when injected into vein 108 by medical device 700, forming implant 736.

In some embodiments, matrix 730 and implant 736 may be formed of a biodegradable polymer mixed with an agent 103. Agent 103 may be substantially similar to agent 103 described with reference to FIG. 1. Matrix 730 and implant 736 may be formed of a biodegradable polymer, or other suitable material that is solid at a human body temperature (e.g., approximately 37 degrees Celsius). That is, matrix 730 may be maintained as a liquid when heated to a first temperature outside of the body, but may solidify when delivered into vein 108 when it comes into contact with venous blood. Implant 736 may degrade over a period of time, e.g., days, weeks, months, years, or another suitable time frame, to deliver agent 103 to lung 122 in a substantially similar manner as described with reference to FIGS. 1 and 2.

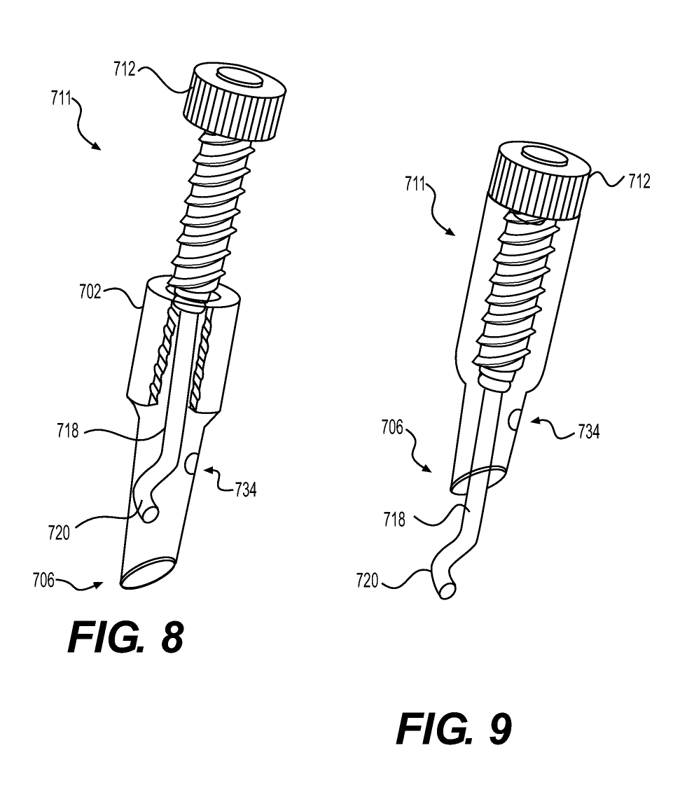

Referring to FIG. 8, actuating assembly 711 is depicted in a first, proximalmost configuration. In the first configuration, distal tip 720 of delivery member 718 may be disposed substantially entirely within lumen 703 of elongate member 702. To deliver an implant 736 to vein 108, an operator may first insert elongate member 702 through tissue and/or incision 106 (referring to FIG. 7) while actuating assembly 711 is in the first configuration. In some embodiments, fluid delivery device 722 may be not be coupled to handle 712 when elongate member 702 is initially inserted into vein 108. After elongate member 702 is disposed through incision 106 and is located within vein 108 (referring to FIG. 7), actuating assembly 711 may be moved to a second, distalmost configuration (referring to FIG. 9) by rotating handle 712 in a first direction (e.g., clockwise or counter clockwise). In the second configuration, distal tip 720 of delivery member 718 may be disposed distal to distal end 706 of elongate member 702. In some embodiments, once actuating assembly 711 is moved to the second configuration, fluid delivery device 722 may be coupled to handle 712.

Once in the second configuration, an operator may then rotate handle 712 in a second direction that is opposite to the first direction (e.g., counter clockwise or clockwise) to simultaneously rotate and longitudinally displace delivery member 718, elongate member 714, and drive member 724 proximally (referring to FIG. 7). Thus, distal tip 720 may move proximally while rotating about longitudinal axis 740, forming a helical, spiral, or corkscrew path. The movement of handle 712 in the second direction may also cause drive member 724 to move proximally, dispensing matrix 730 from fluid delivery device 722 through conduit 732, delivery member 718, and out of distal opening 719. Thus, the movement of handle 712 in the second direction may cause medical device 700 to dispense matrix 730 along the helical, spiral, or corkscrew path of distal tip 720, forming implant 736 within vein 108.

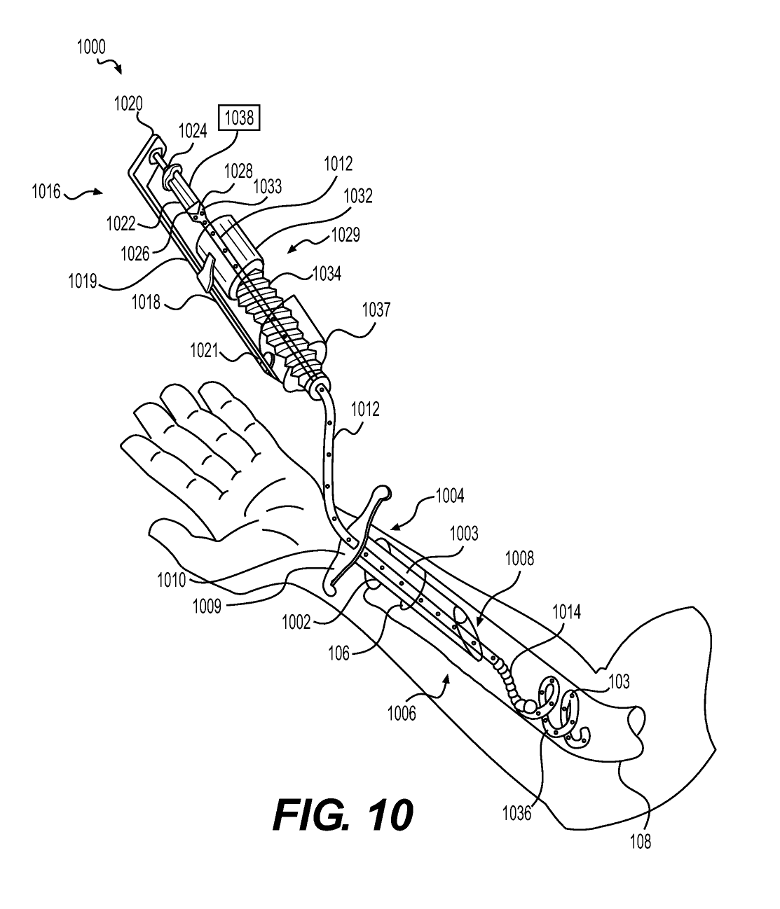

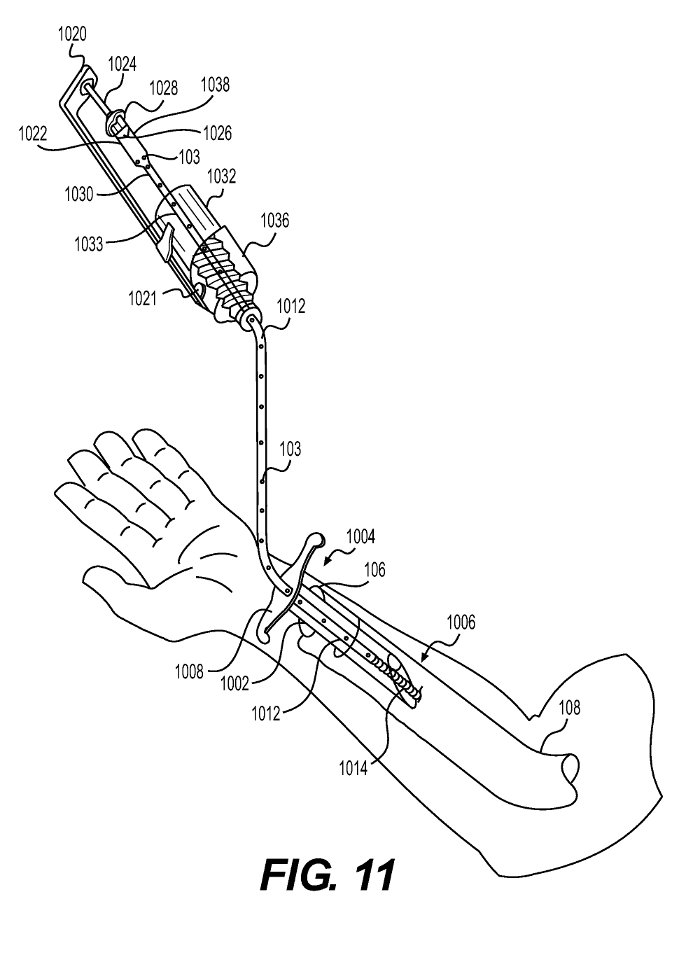

A medical device 1000 configured to deliver an implant 1036 is depicted in FIGS. 10 and 11. Implant 1036 may be mixed with agent 103 and may be substantially similar to implant 736 described with reference to FIG. 7. Medical device 1000 may include an elongate member 1002 having a lumen 1003 that extends from a proximal end 1004 toward a distal end 1006 of elongate member 1002. Elongate member 1002 may include an opening 1008 disposed at distal end 1006. Distal end 1006 of elongate member 1002 may be beveled so that elongate member 1002 can pierce through tissue. In some embodiments, elongate member 1002 may be a needle or other suitable object configured to deliver or withdraw fluids or other substances. Elongate member 1002 may be formed from the similar materials as elongate member 302 described with reference to FIGS. 3-6.

Proximal end 1004 of elongate member 1002 may include a handle 1009 and an opening 1010 disposed within handle 1009. Opening 1010 may be in communication with lumen 1003 of elongate member 1002. A conduit 1012 may be disposed through opening 1010 and through lumen 1003. A distal tip 1014 may extend from and be in fluid communication with a distal end of conduit 1012. Distal tip 1014 may be L-shaped, formed as a spiral, helix, or corkscrew, or may have another suitable shape.

Medical device 1000 may also include a fluid delivery assembly 1016 having a support member 1018. Support member 1018 may be a bracket having an elongate support 1019 and a proximal surface 1020 that is substantially perpendicular to elongate support 1019. Support member 1018 may also include one or more holders 1021 that are configured to hold a fastener 1037 by a friction fit, or the like. Fluid delivery assembly 1016 may include a fluid delivery device 1022, such as, e.g., a syringe or the like. A drive member 1024 may be coupled proximally to end surface 1020, and distally to a stopper 1026 that is configured to dispense a matrix 1030 from fluid delivery device 1022. An actuating assembly 1029 may include an actuator 1032 having a threaded elongate member 1034, and fastener 1037. Actuator 1032 may be a bolt, screw, or other similar member. A lumen 1033 may be disposed through actuator 1032, and may be configured to receive a length of conduit 1012. Threaded elongate member 1034 of actuator 1032 may be configured to mate with fastener 1037 having a threaded bore. Conduit 102 may extend distally from fluid delivery device 1022 through lumen 1033, opening 1010, and lumen 1003. In some embodiments, fluid delivery device 1022 may be coupled to a heater 1038 that is substantially similar to heater 738 described with reference to FIG. 7.

Fluid delivery device 1022 may be coupled to actuator 1032 such that the longitudinal movement of actuator 1032 causes the longitudinal movement of fluid delivery device 1022 in the same direction. In some embodiments, the proximal movement of actuator 1032 may cause the proximal movement of fluid delivery device 1022. The proximal movement of fluid delivery device 1022 may cause drive member 1024 and stopper 1026 to dispense matrix 1030 from fluid delivery device 1022 through conduit 1012.

Referring to FIG. 11, medical device 1000 is depicted in a first, undeployed configuration. In the first configuration, distal tip 1014 may disposed entirely within lumen 1003 of elongate member 1002. In the first configuration, distal tip 1014 may be constrained by lumen 1003 in a substantially straight configuration. To deliver an implant 1036 to vein 108, an operator may first insert elongate member 1002 through tissue and/or incision 106 while medical device 1000 is in the first configuration. In some embodiments, actuator 1032 may be disposed in a distalmost configuration when medical device 1000 is in the first configuration. After elongate member 1002 is disposed through incision 106 and is located within vein 108, an operator may retract only elongate member 1002 proximally, leaving distal tip 1014 within vein 108 as depicted in FIG. 10.

Once distal tip 1014 is in the unconstrained configuration of FIG. 10, actuator 1032 may be moved proximally by rotating actuator 1032 in a first direction (e.g., clockwise or counter clockwise). The rotation of actuator 1032 may simultaneously rotate and longitudinally displace distal tip 1014, elongate member 1034, and fluid delivery device 1022 proximally. Thus, distal tip 1014 may move proximally while rotating about a longitudinal axis, forming a helical, spiral, or corkscrew path. Drive member 1024 and stopper 1026 may remain stationary during the proximal movement of fluid delivery device 1022, causing drive member 1024 and stopper 1026 to dispense matrix 1030 from fluid delivery device 1022 through conduit 1012, and out of distal tip 1014. Thus, the movement of actuator 1032 in the first direction may cause medical device 1000 to dispense matrix 1030 along the helical, spiral, or corkscrew path of distal tip 1014, forming implant 1036 within vein 108.

In some embodiments, medical device 700 may be modified such that fluid delivery device 722 dispenses fluid distally through actuating assembly 711 toward delivery member 718 in a manner substantially similar to the mechanism depicted in FIGS. 10 and 11. In other embodiments, medical device 1000 may be modified such that fluid delivery device 1022 dispenses fluid proximally via a conduit that is coupled with distal tip 1014 in a manner substantially similar to the mechanism depicted in FIG. 7.

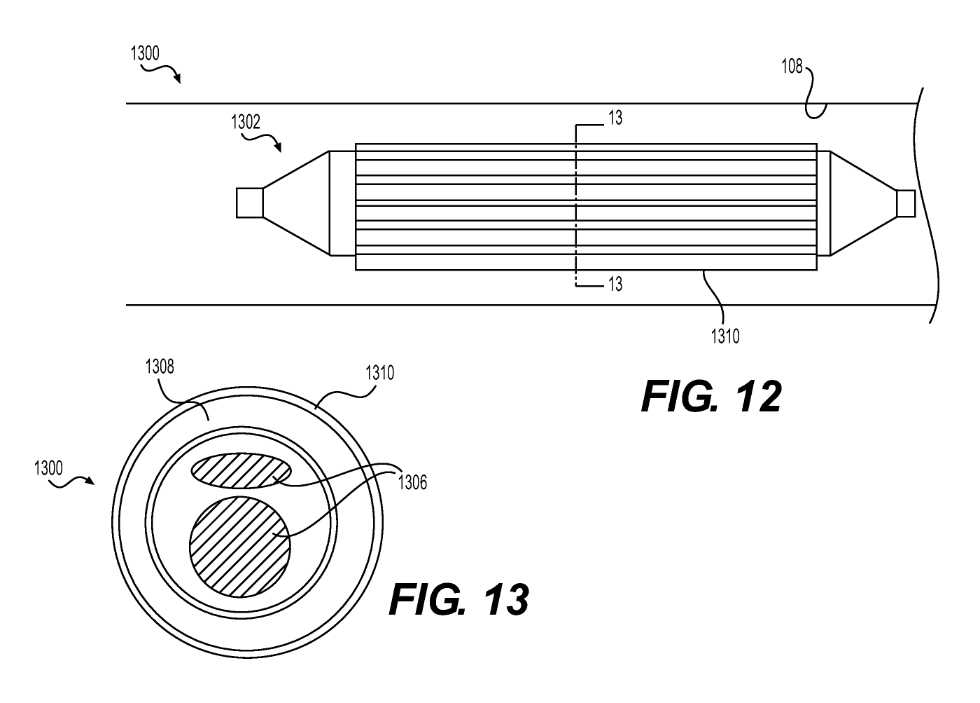

A medical device 1300 is depicted in FIGS. 12 and 13. Medical device 1300 may be an attachment that is coupled to the distal end of a catheter that can extend from an elongate member such as, e.g., a bronchoscope (not shown). Medical device 1300 may include a casing 1302 having a plurality of lumens 1306 disposed within an expandable member 1308. Expandable member 1308 may be a balloon or other suitable expandable device known in the art. Lumens 1306 may be configured to inflate and deflate expandable member 1308 from a deflated configuration to an inflated configuration. An implant 1310 may be disposed on an outer surface of expandable member 1308. Implant 1310 may be cylindrical or another suitable shape (e.g., a drug-eluting stent), and may elute agent 103 into the bloodstream. In some embodiments, implant 1310 may be substantially similar to implant 100 described with reference to FIG. 1. In some embodiments, implant 1310 may include a bioadhesive as described with reference to FIG. 1 in order to adhere to the inner surface of vein 108. In some embodiments, implant 1310 may be sprayed or otherwise placed onto the outer surface of expandable member 1308 by mechanisms known in the art. For example, implant 1310 can be an agent that can elute from a balloon. Medical device 1300 may also be similar to, and incorporate features from drug eluting medical devices described in U.S. Patent Application Publication 2012/0095396 A1 that published on Apr. 19, 2012, the entirety of which is incorporated by reference herein.

Medical device 1300 may be inserted into vein 108 in the deflated configuration as shown in FIG. 12. Medical device 1300 may be expanded to the inflated configuration such that implant 1310 is caused to contact the inner surface of vein 108. The bioadhesive incorporated or otherwise coated onto the outer surface of implant 1310 may adhere to the inner wall of vein 108. Once, implant 1310 is secured to the vein 108, lumens 1306 may direct the expansion fluid out of medical device 1300 to move medical device 1300 from the inflated configuration to the deflated configuration, so that medical device 1300 may be removed from vein 108. Once implanted within vein 108, implant 1310 may deliver agent 103 (shown in FIG. 1) to lungs 122 as described with reference to FIG. 1.

Any aspect set forth in any embodiment may be used with any other embodiment set forth herein. The devices and apparatus set forth herein may be used in any suitable medical procedure, may be advanced through any suitable body lumen and body cavity, and may be used to remove material from any suitable body portion. For example, the apparatuses and methods described herein may be used through any natural body lumen or tract, or through incisions in any suitable tissue.

It will be apparent to those skilled in the art that various modifications and variations can be made in the disclosed systems and processes without departing from the scope of the invention. Other embodiments of the invention will be apparent to those skilled in the art from consideration of the specification and practice of the invention disclosed herein. It is intended that the specification and examples be considered as exemplary only. The following disclosure identifies some other exemplary embodiments.

We claim:

1. A method of treating a disease of the lung, the method comprising:
inserting an implant into the circulatory system, wherein the implant includes an agent configured to be released into the circulatory system over time to deliver the agent to the lungs, the agent being configured to treat a disease of the lung.

2. The method of claim 1, wherein the agent is released into the bloodstream, and the agent enters the lung through an exchange between one or more capillaries of the circulatory system and one or more alveoli of the lung.

3. The method of claim 1, wherein the agent is configured to treat one or more of COPD, bronchitis, emphysema, asthma, an allergy of the lung, and lung cancer.

4. The method of claim 1, wherein the implant is configured to gradually degrade over a period of months to release the agent.

5. The method of claim 1, wherein the implant is inserted into a vein that leads to the right atrium of the heart.

6. The method of claim 1, wherein the implant is polymeric.

7. The method of claim 1, wherein inserting the implant into the circulatory system further includes:
the implant coupled to an expandable member, the expandable member being movable between a retracted configuration and an expanded configuration;
inserting the expandable member and the implant into the circulatory system while the expandable member is in the retracted configuration; and
expanding the expandable member to the expanded configuration to adhere the implant to an inner surface in the circulatory system.

8. A medical device, comprising:
a first elongate member having a lumen extending between a proximal end and a distal end of the first elongate member;
a second elongate member disposed through the first elongate member, the second elongate member including:
a handle portion disposed at a proximal end; and
a lumen disposed through the second elongate member and the handle portion;
a plunger disposed in the second elongate member, the plunger including:
a proximal portion; and
a third elongate member extending distally from the proximal portion; and
an implant disposed within the second elongate member distal to the plunger, wherein the implant includes an agent configured to be released into the circulatory system over time to deliver the agent to the lungs, the agent being configured to treat a disease of the lung.

9. The medical device of claim 8, further including a flange disposed at the proximal end of the first elongate member, wherein, in a first configuration, the second elongate member is disposed at a proximal position such that:
a distal end of the second elongate member is disposed within the first elongate member; and
the handle portion of the second elongate member is disposed proximal to the flange of the first elongate member by a first distance.

10. The medical device of claim 9, wherein, in a second configuration, the second elongate member is disposed at a distal position such that:
the distal end of the second elongate member is disposed distal to the distal end of the first elongate member;
an entirety of the implant is disposed distal to the distal end of the first elongate member;
the second elongate member, the plunger, and the implant are disposed distally by approximately the first distance relative to their respective positions in the first configuration; and
the handle portion of the second elongate member and the flange of the first elongate member are adjacent to one another.

11. The medical device of claim 10, wherein, in a third configuration:
the plunger and the implant are disposed in the substantially same positions as the second configuration; and
the second elongate member is disposed proximal to the distal position of the second elongate member to remove the implant from the distal end of the second elongate member.

12. A medical device, comprising:
a first elongate member having a lumen extending between a proximal end and a distal end;
a fluid delivery device containing an agent, the agent configured to be dispensed into the circulatory system over time to deliver the agent to the lungs, the agent being configured to treat a disease of the lung;
an actuator coupled to the first elongate member and the fluid delivery device; and
a distal tip coupled to the fluid delivery device, wherein the proximal movement of the actuator moves the distal tip longitudinally and dispenses agent from the distal tip.

13. The medical device of claim 12, further including a heater coupled to the fluid delivery device configured to maintain the agent in liquid form.

14. The medical device of claim 13, wherein the fluid delivery device further contains a matrix, and wherein the matrix and the agent are configured to be in solid form at a human body temperature.

15. The medical device of claim 12, wherein the proximal movement of the actuator moves the distal tip along and about a longitudinal axis in a spiral path.

16. The medical device of claim 12, wherein:
the actuator is an elongate member having a thread; and
the first elongate member further includes a bore configured to receive the actuator.

17. The medical device of claim 16, further including a drive member coupled to a proximal end of the actuator, wherein the proximal movement of the actuator causes the drive member to dispense the agent from the fluid delivery device and through the distal tip.

18. The medical device of claim 12, further including a conduit extending from the fluid delivery device, through the first elongate member, and in fluid communication with the distal tip.

19. The medical device of claim 18, wherein:
in a first configuration, the distal tip is constrained within the first elongate member; and
the first elongate member is configured to retract relative to the distal tip to remove the distal tip from the first elongate member in a second configuration.

20. The medical device of claim 18, wherein:
the actuator includes an elongate member having an elongate thread;
the medical device further includes a fastener configured to receive the actuator; and
the conduit further extends through the actuator.
